(19) United States
(12) Patent Application Publication
(10) Pub. No.: US 2009/0191144 A1
(43) Pub. Date: Jul. 30, 2009

(54) USE OF LIPID COMPONENTS AGAINST POLLEN ALLERGIES

(75) Inventors: Rainer KROEPKE, Schenefeld (DE); Kerstin Bohnsack, Hamburg (DE); Frank Rippke, Hamburg (DE); Alexander Filbry, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 12/360,684

(22) Filed: Jan. 27, 2009

(30) Foreign Application Priority Data
Jan. 28, 2008 (DE) 10 2008 006 393.2

(51) Int. Cl. A61K 31/215; A61K 31/765; A61K 31/01; A61P 37/08

(52) U.S. Cl. 424/78.02; 514/529; 514/762

(57) ABSTRACT

A cosmetic or dermatological preparation for combating or substantially preventing pollen allergies. The preparation comprises or essentially consists of a lipid phase which comprises one or more lipids and has a spreading coefficient of less than about 800 mm²/10 min at 25° C. and a viscosity of from about 40 to about 25,000 mPas. This Abstract is not intended to define the invention disclosed in the specification, nor intended to limit the scope of the invention in any way.

### CROSS-REFERENCE TO RELATED APPLICATIONS

The present application claims priority under 35 U.S.C. § 119 of German Patent Application No. 10 2008 006 393.2, filed Jan. 28, 2008, the entire disclosure of which is expressly incorporated by reference herein.

### BACKGROUND OF THE INVENTION

1. Field of the Invention

The present invention relates to the use of certain lipid components in cosmetic or dermatological preparations against pollen allergies.

2. Discussion of Background Information

Pollen from certain trees, grasses, grain types and herbs causes among those affected a running or stuffy nose, itchy eyes and constant sneezing.

The pollen or farina is formed in the anther of seed plants. It is composed of the pollen grains, the microspores. The pollen grains are surrounded by a tough cell wall, which is composed, among other things, of sporopollenin.

Pollen grains serve to bring the male spores protected to the female reproductive structure and thus to ensure the pollination and subsequently the fertilization.

Allergic reactions to plant pollen are becoming an increasing problem. Allergies and hypersensitivity can manifest themselves:

On the mucous membranes (allergic rhinitis (hay fever), swelling of the oral mucosa, conjunctivitis)
In the respiratory system (bronchial asthma)
On the skin (atopic dermatitis (neurodermatitis), contact dermatitis, urticaria)
In the gastro-intestinal tract (nausea, diarrhea, particularly among infants and toddlers)
As an acute emergency (anaphylactic shock).

Allergy sufferers can suffer from one form of the disease, but also from mixed forms. While allergic symptoms typically tend to occur acutely on the mucous membranes, symptoms such as bronchial asthma and atopic dermatitis can take a chronic course.

First signs of pollen allergy can occur among children from the age of five to six. Hay fever is a problem for children and their parents. Children afflicted by hay fever suffer from itchy and watery eyes, running nose, breathing problems and fatigue. Because there is additionally the danger that hay fever will turn into bronchial asthma, consistent pollen protection and preventive measures are very important.

Antihistamines and cortisone can be used to alleviate acute symptoms. The new hay fever remedies with an antihistamine as active ingredient are very reliable and, in contrast to their predecessors, do not cause much fatigue. Nevertheless, adults should be careful when driving vehicles or operating machinery.

Pollen flight can furthermore trigger or intensify neurodermatitis. Allergens in the air can trigger not only asthma and hay fever, but also reactions on the skin. The latest studies using a test developed at the Technical University of Munich (Prof. Johannes Ring) showed that pollen from birch, hazel, alder, grasses and herbs triggered eczema on the skin of certain people—and neurodermatitis is ultimately an eczema. People are also thereby affected on the skin by this type of pollen allergy who do not suffer from hay fever or other allergies.

It was disputed for a long time that allergies play a role in the development of neurodermatitis—it was assumed to be a congenital disease that is intensified by psychological factors.

It has now surprisingly been found that the use of lipids or lipid mixtures for the manufacture of a cosmetic or dermatological preparation for combating or substantially preventing pollen allergies wherein the spreading coefficient of the lipid phase of the preparation is less than about 800 mm²/10 min at 25° C. and the viscosity of the lipids/lipid phase is in the range of from about 40 to about 25,000 mPas is suitable for avoiding or at least alleviating pollen allergies.

### SUMMARY OF THE INVENTION

The present invention provides a cosmetic or dermatological preparation for combating or substantially preventing pollen allergies. The preparation comprises or essentially consists of a lipid phase which comprises one or more lipids and has a spreading coefficient of less than about 800 mm²/10 min at 25° C. and a viscosity of from about 40 to about 25,000 mPas.

In one aspect of the preparation, the lipid phase may have a spreading coefficient of less than about 600 mm²/10 min at 25° C., e.g., less than about 400 mm²/10 min at 25° C. and/or the lipid phase may have a viscosity of from higher than about 80 to about 15,000 mPas, e.g., from higher than about 100 to about 8,000 mPas.

In another aspect, the preparation of the present invention may comprise one or more stabilizers, for example, at least one substance selected from acetyltrifluoromethylphenylvalylglycine, acrylamide ammonium acrylate copolymer, aluminum magnesium hydroxide stearate, ammonium lactate, ammonium polyacrylate, ammonium polyacryloyldimethyl taurate, arginine PCA, capryloyl salicylic acid ester, cinnamic acid, cocoglucoside, copper gluconate, diphenyldimethicone, disodium adenosine triphosphate, disodium succinate, disteardimonium hectorite, dodecene, eperua falcata, hydrogenated palm glyceride, hydrogenated palm glyceride citrate, hydrogenated palm kernel glycerides, hydrolyzed wheat protein, PG propyl methyl silanediol, hydroxyethyl acrylate/sodium acryloyldimethyltaurate copolymer, isodeceth-6, linseed acid magnesium aspartate, melibiose, oxothiazolidine carboxylic acid, palmitoyl pentapeptide 4, PEG-8 laurate, phenethylalcohol, phenyl propanol, polyacrylate-13, polyacrylate-3, sarcosine, saxifraga sarmentosa extract, scutellaria baicalensis extract, sodium metabisulfite, soybean isoflavone, tocopherylglucoside, trideceth-6, and zinc gluconate.

In yet another aspect, the one or more stabilizers may be present in a concentration of from about 0.1% to about 30% by weight, based on the total weight of the preparation, for example, in a concentration of from about 0.5% to about 10% by weight, or from about 1.0% to about 6.0% by weight.

In a still further aspect of the preparation, the one or more lipids may comprise at least one lipid selected from medicinal white oils, esters of C8-C30 fatty acids and linear and branched C8-C24 alcohols, and linear silicone oils.

In another aspect, the preparation may have a pH of from about 4 to about 6, e.g., form about 4.6 to about 5.4 and/or the preparation may be present as an emulsion, e.g., an emulsion comprising at least about 5% by weight of water, and/or the preparation may be present as a solid stick, an ointment or an aerosol.

The present invention also provides a method of combating or substantially preventing pollen allergies. The method comprises applying to skin a cosmetic or dermatological preparation according to the present invention as set forth above (including the various aspects thereof).

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description making apparent to those skilled in the art how the several forms of the present invention may be embodied in practice. Unless stated otherwise, all amounts, fractions and percentages given are based on the weight and the total amount or on the total weight of the preparations.

Measuring the spreading coefficient (in mm²/10 min) referred to above is carried out according to the following method. 20 μl of the substance to be tested is dripped centrally on a Rotband filter paper from Schleicher & Schüll, Germany. At the same time a stopwatch is started and after 10 min the area is measured that has been wetted by the substance during this time. The measurement is carried out in an isothermal room at 25° C.+/−1° C.

Preparations of the present inventions are capable of substantially preventing or avoiding allergic reactions to pollen among the people affected and provide the patient or the user of a preparation of this type with relief from allergic reactions at an early stage.

Preferred lipids include:

| | | Viskosity (in mPas) |
|---|---|---|
| Trade name | INCI Name | (s⁻¹ = 500) |
| Avocado oil refined DAC | Persea Gratissima | 180 |
| Shell Ondina 4222 | Mineral Oil | 43.9 |
| Myritol 331 | Cocoglycerides | 41 |
| Crodamol PTIS 3797 | Pentaerythrityl Tetraisostearate | 390 |
| Pionier 6301 | Mineral Oil | 51 |
| Eutanol G | Octyldodecanol | 47.7 |
| Miglyol 829 | Caprylic/Capric/Diglyceryl Succinate | 117 |
| Cosmacol ETI | Di-C12/13 Alkyl Tartrate | 107 |
| Pionier 2071 | Mineral Oil | 98 |
| Silkflo 366 NF | Polydecene | 45 |
| Abil Wax 9840 | Cetyl Dimethicone | 171 |
| Jojoba Oil FP35 Golden | Buxus Chinensis | 150 |
| Uniphen P-23 | Phenoxyethanol, Methylparaben, | 54.3 |
| | Ethylparaben, Propylparaben, Butylparaben, | |
| | Isobutylparaben | |
| Fluilan | Lanolin Oil | 3,240 |
| Parsol MCX | Ethylhexyl Methoxycinnamate | 81.2 |
| Macadamia nut oil | Macadamia Ternifolia | 180 |
| Motif | Squalan | 220 |
| Lipovol MOS-130 | Tridecyl Stearate(+) Tridecyl Trimellitate(+) | 47 |
| | Dipentaerythrityl Hexacaprylate/Hexacaprate | |
| Rewopal PIB 1000 | Polyisobutene | 22,170 |
| Polysynlan | Hydrogenated Polyisobutene | 57 |
| Prisorine 2041 GTIS | Triisostearin | 99 |
| Castor Oil PH. EUR | Ricinus Communis | 820 |
| Wacker AK 100 | Dimethicone | 101 |
| Ucon Fluid AP | PPG-14 Butyl Ether | 96 |
| Crodamol BS | Butyl Stearate | 100 |
| Dermol 488 | PEG-2 Diethylhexanoate | 110 |
| Dermosoft MCA | Dipropylene Glycol + Caprylyl Glycol + | 65 |
| | Glyceryl Laurate + Phenylpropanol | |
| | Hydroxystearate Glycerol Triacetyle | 65 |
| DUB DIAMOND | C10-40 Isoalkyl Acid Triglycerides | 132 |
| DUB MDIS | Diisostearyl Malate | 113 |
| DUB OLIOSE | Cannabis Sativa Seed Oil | 46 |
| DUB PTIS | Pentaerythrityl Tetraisostearate | 57 |
| DUB TMI | Triisodecyl Trimelliate | 111 |
| DUB TMTD | Tridecyl Trimellitate | 116 |
| Emerest 2384 | Propylene Glycol Monoisostearate | 42 |
| Hostaphat B 310 | Tributoxyethylphosphate | 122 |
| Hydrobrite 1000 PO | Hydrogenated Petrolatum | 568 |
| Isodragol | Triisononanoin | 67 |
| Lipex Olive | Olive Oil | 78 |
| Mobil PureSyn 1000 | Hydrogenated C6-14 Olefin Polymers | 3,450 |
| Trivent PE 48 | Pentaerythrityl Tetraethylhexanoate | 116 |
| Wacker Belsil 1000 | Dimethicone | 1,177 |
| Wacker Belsil 2000 | Dimethicone | 2,306 |
| Wacker Belsil 350 | Dimethicone | 480 |
| Wacker Belsil 500 | Dimethicone | 585 |
| Wacker Silikonöl AK 50 | Polydimethylsiloxane | 51 |

The viscosity of the lipid phase of a preparation of the present invention can be increased by rheological modifiers and lipid thickeners. Preferred lipid thickeners include hectorite, silicate (“Aerosil 972”), metal salts of stearic acid, hydrogenated castor oil, microwaxes having a melting point above about 42° C., paraffins having a melting point above about 42° C., native waxes having a melting point of from about 33° C. to about 78° C., glyceryl esters of isostearates, fillers with a specific surface area of at least about 0.2 m²/g, long-chain silicone oils, bentonites and/or modified bentonites.

The cosmetic or dermatological preparations according to the present invention can be composed as usual and be used for the treatment, the care and the cleansing of the skin and/or the hair and as a cosmetic product in decorative cosmetics.

They often will comprise from about 0.001% to about 10% by weight, preferably from about 0.05% to about 5% by weight, in particular from about 0.1% to about 2% by weight, based on the total weight of the preparation, of 3-(4-hydroxy-3-methoxyphenyl)-1-(4-hydroxyphenyl)-propane-1-one.

Cosmetic and dermatological preparations according to the present invention can be present in various forms. They can be, for example, a solution, an anhydrous preparation, an emulsion or a microemulsion of the type water-in-oil (W/O), or the type oil-in-water (O/W), a multiple emulsion, for example of the type water-in-oil-in-water (W/O/W), a gel, a solid stick, an ointment or also an aerosol.

It is also advantageous according to the present invention to administer 3-(4-hydroxy-3-methoxyphenyl)-1-(4-hydroxyphenyl)-propane-1-one in encapsulated form, for example, in collagen matrices and other conventional encapsulation materials such as, e.g., as cellulose encapsulations, in gelatin, wax matrices or liposomally encapsulated. In particular wax matrices such as are described in DE-OS 43 08 282, the entire disclosure whereof is incorporated by reference herein, have proven to be advantageous.

It is also possible and advantageous according to the present invention to incorporate 3-(4-hydroxy-3-methoxyphenyl)-1-(4-hydroxyphenyl)-propane-1-one into aqueous systems or surfactant preparations for cleansing the skin and the hair.

The preparation of the present invention may further comprise an active substance complex of panthenol, glycerol and at least one of citrate and bisabolol, wherein the mass ratio of panthenol to citrate is from about 25:1 to about 5:1, based on the citrate anion, and/or the mass ratio of panthenol to bisabolol is from about 5:1 to about 1:1. Further, in this case it is preferred for the mass ratio of glycerol to citrate to be from about 60:1 to about 10:1, based on the citrate anion, and/or for the mass ratio of glycerol to bisabolol to be from about 50:1 to about 1:1.

The cosmetic and dermatological preparations according to the present invention can comprise cosmetic auxiliaries, for example, those which are customarily used in such preparations such as, e.g., preservatives, bactericides, perfumes, substances for preventing foaming, dyes, pigments that have a coloring action, thickeners, surface-active substances, emulsifiers, softening, moisturizing and/or humectant substances, fats, oils, waxes or other customary constituents of a cosmetic or dermatological formulation, such as alcohols, polyols, polymers, gel formers, foam stabilizers, electrolytes, organic solvents or silicone derivatives.

Advantageous gel formers for the preparations of the present invention include, for example, copolymers of C10-30-alkyl acrylates and one or more monomers of acrylic acid, of methacrylic acid, and/or esters thereof. The INCI name of such compounds is “Acrylates/C 10-30 Alkyl Acrylate Cross-polymer.” The PEMULEN® grades TR 1, TR 2 and TRZ from Goodrich (Noveon) are particularly advantageous.

Carbopols are also advantageous gel formers for such preparations. Carbopols are polymers of acrylic acid, in particular also acrylate-alkyl acrylate copolymers. Advantageous carbopols are, for example, the grades 980, 981, 984, 1342, 1382, 2984 and 5984, likewise the ETD grades 2001, 2020, 2050 and Carbopol Ultrez 10, PVM/MA decadiene crosspolymer (trade name STABILEZE® 06), polyglyceryl methacrylate and polyacrylamide, ammonium dimethyl tauramide/vinylformamide copolymers, copolymers or crosspolymers comprising acryloyl dimethyltaurate, polyacryloyl dimethyltauramide, polyvinylpyrrolidone, and copolymers thereof.

Further advantageous gel formers for such preparations include xanthan gum, polyvinylpyrrolidone, cellulose derivatives, in particular, cellulose ethers such as, e.g., hydroxypropyl methylcellulose, starch and starch derivatives, hyaluronic acid, carrageenan, silicon dioxide and aluminum silicates.

The quantity of antioxidants (one or more compounds) in the preparations is preferably from about 0.001% to about 30% by weight, particularly preferably from about 0.05% to about 20% by weight, in particular from about 1% to about 10% by weight, based on the total weight of the preparation.

The lipid phase can advantageously be chosen from the following substances:
Mineral oils, mineral waxes
Oils, such as triglycerides of capric acid and/or caprylic acid, and also natural oils, such as, for example, castor oil,
Fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids;
Alkyl benzoates;
Silicone oils, such as dimethicones, cyclomethicones, dimethylpolysiloxanes, diethylpolysiloxanes, diphenylpolysiloxanes, and mixed forms thereof.

The oil phase of the emulsions, oleogels or hydrodispersions or lipodispersions of the present invention is advantageously selected from the esters of saturated and/or unsaturated, branched and/or unbranched alkanecarboxylic acids with a chain length of from about 3 to about 30 carbon atoms, and saturated and/or unsaturated, branched or unbranched alcohols with a chain length from about 3 to about 30 carbon atoms, and from the esters of aromatic carboxylic acids and saturated and/or unsaturated, branched and/or unbranched alcohols with a chain length of from about 3 to about 30 carbon atoms. Such ester oils can advantageously be selected from isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, as well as synthetic, semisynthetic, and natural mixtures of such esters, for example, jojoba oil.

Optionally, the aqueous phase of the preparations of the present invention advantageously comprises alcohols, diols, or polyols having a low number of carbon atoms, as well ethers thereof, preferably ethanol, isopropanol, propylene glycol, glycerin, ethyleneglycol, ethyleneglycol monoethyl or monobutyl ether, propyleneglycol mono methyl, monoethyl, or monobutyl ether, diethyleneglycol monomethyl or monoethyl ether, and analogous products, furthermore alcohols having a low number of carbon atoms, for example, ethanol, isopropanol, 1,2-propanediol, glycerin, as well as in particular one or more thickeners, which can be advantageously chosen from silicon dioxide, aluminum silicates, polysaccharides or derivatives thereof, e.g., hyaluronic acid, xanthan gum, hydroxypropyl methylcellulose, particularly preferably from the group of polyacrylates, preferably polyacrylates from the group of the so-called carbopols, for example carbopols grades 980, 981, 1382, 2984, 5984, in each case individually or in combination.

In particular mixtures of the above-referenced solvents may be used. In the case of alcoholic solvents, water can be a further constituent.

Emulsions according to the present invention are advantageous and comprise, e.g., the cited fats, oils, waxes and other fatty bodies, as well as water and one or more emulsifiers, such as, e.g., emulsifiers which are conventionally used for such a type of formulation.

Gels according to the present invention usually comprise alcohols having a low number of carbon atoms, e.g., ethanol, isopropanol, 1,2-propanediol and glycerol, and water or an above-referenced oil in the presence of a thickener that with oily alcoholic gels is preferably silicon dioxide or an aluminum silicate, and with aqueous-alcoholic or alcoholic gels is preferably a polyacrylate.

Suitable propellants for preparations of the present invention which can be sprayed from aerosol containers include the customary known readily volatile, liquefied propellants, for example hydrocarbons (propane, butane, isobutane), which can be employed individually or as mixtures. Compressed air can also advantageously be used.

Preparations according to the present invention can also advantageously comprise substances that absorb UV radiation in the UVA and/or UVB range, wherein the total concentration of the filter substances is, e.g., from about 0.1% by weight to about 30% by weight, preferably from about 0.5% to about 10% by weight, in particular from about 1.0% to about 6.0% by weight, based on the total weight of the preparation, in order to provide preparations that protect the hair or the skin from the entire range of ultraviolet radiation. They can also be used as sun block for the hair or the skin.

Preparations according to the present invention can also advantageously comprise substances that act as a stabilizer, wherein the total amount of these stabilizers is, e.g., from about 0.1% to about 30% by weight, preferably from about 0.5% to about 10% by weight, in particular from about 1.0% to about 6.0% by weight, based on the total weight of the preparation, in order to provide cosmetic preparations that render possible preparations with long-term stability.

Preferred stabilizers include substances from the group of acetyltrifluoromethylphenylvalylglycine, acrylamide ammonium acrylate copolymer, aluminum magnesium hydroxide stearate, ammonium lactate, ammonium polyacrylate, ammonium polyacryloyldimethyl taurate, arginine PCA, capryloyl salicylic acid ester, cinnamic acid, cocoglucoside, copper gluconate, diphenyldimethicone, disodium adenosine triphosphate, disodium succinate, disteardimonium hectorite, dodecene, eperua falcata, hydrogenated palm glyceride, hydrogenated palm glyceride citrate, hydrogenated palm kernel glyceride, hydrolyzed wheat protein, PG propyl methyl silanediol, hydroxyethylacrylate/sodium acryloyldimethyltaurate copolymer, isodeceth-6, linseed acid magnesium aspartate, melibiose, oxothiazolidine carboxylic acid, palmitoyl pentapeptide 4, PEG-8 laurate, phenethylalcohol, phenyl propanol, polyacrylate-13, polyacrylate-3, sarcosine, saxifraga sarmentosa extract, scutellaria baicalensis extract, sodium metabisulfite, soybean isoflavone, tocopheryl glucoside, trideceth-6, and zinc gluconate.

The following examples are intended to illustrate the present invention without limiting it. Unless stated otherwise, all of the quantities, proportions and percentages are percentages by weight based on the weight and the total quantity or the total weight of the preparations.

| Example No. | 1 | 2 | 3 |
|---|---|---|---|
| Cyclic silicone oil | | | 3 |
| Sorbitanstearate | | | 2 |
| Lanolin alcohol | 3 | | |
| Polyglyceryl-2 dipolyhydroxystearate | | 3 | |
| Polyglyceryl-3 diisostearate | | 2 | |
| Cetyl palmitate | | | 10 |
| Vitamin E acetate | | 1 | 0.5 |
| Propylparaben | | 0.15 | 0.1 |
| Methylparaben | | 0.4 | 0.3 |
| Microwax | 15 | | |
| Microwax + medicinal white oil | 41 | | |
| Medicinal white oil | 38.75 | 10 | 6 |
| Medicinal white oil, low-viscosity | | | 2 |
| Glycerin | 1 | 8.7 | 10 |
| Cetyl alcohol | | | 3 |
| Aluminum salt of starch octenyl- | | | 1.5 |
| succinic acid | | | |
| Isopropyl stearate | | 11.25 | |
| Perfume | | 0.15 | 0.15 |
| Citric acid | | 0.086 | 0.086 |
| Sodium citrate | | 0.174 | 0.174 |
| Magnesium sulfate | | 0.6 | |
| Bisabolol | 0.25 | | |
| Carbomer, sodium salt | | | 0.28 |
| Phenoxyethanol | | 0.8 | 0.8 |
| Cetearyl alcohol | | | |
| PEG-150 Distearate | | | |
| Panthenol | 1 | 4 | 6.7 |
| Water | | ad 100 | ad 100 |
| pH value | | 5 +/− 0.7 | 5 +/− 0.7 |

While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A cosmetic or dermatological preparation for combating or substantially preventing pollen allergies, wherein the preparation comprises or essentially consists of a lipid phase which comprises one or more lipids and has a spreading coefficient of less than about 800 mm²/10 min at 25° C. and a viscosity of from about 40 to about 25,000 mPas.

2. The preparation of claim 1, wherein the lipid phase has a spreading coefficient of less than about 600 mm²/10 min at 25° C.

3. The preparation of claim 1, wherein the lipid phase has a spreading coefficient of less than about 400 mm²/10 min at 25° C.

4. The preparation of claim 1, wherein the lipid phase has a viscosity of from higher than about 80 to about 15,000 mPas.

5. The preparation of claim 2, wherein the lipid phase has a viscosity of from higher than about 80 to about 15,000 mPas.

6. The preparation of claim 1, wherein the lipid phase has a viscosity of from higher than about 100 to about 8,000 mPas.

7. The preparation of claim 3, wherein the lipid phase has viscosity of from higher than about 100 to about 8,000 mPas.

8. The preparation of claim 1, wherein the preparation further comprises one or more stabilizers.

9. The preparation of claim 8, wherein the one or more stabilizers comprise at least one substance selected from acetyltrifluoromethylphenylvalylglycine, acrylamide ammonium acrylate copolymer, aluminum magnesium hydroxide stearate, ammonium lactate, ammonium polyacrylate, ammonium polyacryloyldimethyl taurate, arginine PCA, capryloyl salicylic acid ester, cinnamic acid, cocoglucoside, copper gluconate, diphenyldimethicone, disodium adenosine triphosphate, disodium succinate, disteardimonium hectorite, dodecene, eperua falcata, hydrogenated palm glyceride, hydrogenated palm glyceride citrate, hydrogenated palm kernel glycerides, hydrolyzed wheat protein, PG propyl methyl silanediol, hydroxyethyl acrylate/sodium acryloyldimethyltaurate copolymer, isodeceth-6, linseed acid magnesium aspartate, melibiose, oxothiazolidine carboxylic acid, palmitoyl pentapeptide 4, PEG-8 laurate, phenethylalcohol, phenyl propanol, polyacrylate-13, polyacrylate-3, sarcosine, saxifraga sarmentosa extract, scutellaria baicalensis extract, sodium metabisulfite, soybean isoflavone, tocopherylglucoside, trideceth-6, and zinc gluconate.

10. The preparation of claim 8, wherein the one or more stabilizers are present in a concentration of from about 0.1% to about 30% by weight, based on a total weight of the preparation.

11. The preparation of claim 10, wherein the concentration is from about 0.5% to about 10% by weight.

12. The preparation of claim 1, wherein the one or more lipids comprise at least one lipid selected from medicinal white oils, esters of C8-C30 fatty acids and linear and branched C8-C24 alcohols, and linear silicone oils.

13. The preparation of claim 1, wherein the preparation has a pH of from about 4 to about 6.

14. The preparation of claim 1, wherein the preparation is present as an emulsion.

15. The preparation of claim 14, wherein the preparation comprises at least about 5% by weight of water, based on a total weight of the preparation.

16. The preparation of claim 1, wherein the preparation is present as at least one of a solution, an anhydrous preparation, a solid stick, an ointment and an aerosol.

17. A method of combating or substantially preventing pollen allergies, wherein the method comprises applying to skin a cosmetic or dermatological preparation which comprises or essentially consists of a lipid phase which comprises one or more lipids and has a spreading coefficient of less than about 800 mm²/10 min at 25° C. and a viscosity of from about 40 to about 25,000 mPas.

18. The method of claim 17, wherein the one or more lipids comprise at least one lipid selected from medicinal white oils, esters of C8-C30 fatty acids and linear and branched C8-C24 alcohols, and linear silicone oils.

19. The method of claim 17, wherein the lipid phase has at least one of a spreading coefficient of less than about 600 mm²/10 min at 25° C. and a viscosity of from higher than about 80 to about 15,000 mPas.

20. The method of claim 18, wherein the lipid phase has at least one of a spreading coefficient of less than about 400 mm²/10 min at 25° C. and a viscosity of from higher than about 100 to about 8,000 mPas.
